(19) United States
(12) Patent Application Publication
(10) Pub. No.: US 2006/0128617 A1
(43) Pub. Date: Jun. 15, 2006

(54) Oligoribonucleotide or peptidic nucleic acid inhibiting function of hepatitis c virus

(75) Inventors: Michinori Kohara, Tokyo (JP); Tsunamasa Watanabe, Tokyo (JP); Kazunari Taira, Ibaraki (JP); Makoto Miyagishi, Chiba (JP); Masayuki Sudo, Kanagawa (JP)

Correspondence Address: DAVIDSON, DAVIDSON & KAPPEL, LLC, 485 SEVENTH AVENUE, 14TH FLOOR, NEW YORK, NY, 10018, US

(73) Assignees: TOKYO METROPOLITAN ORGANIZATION FOR MEDICAL RESEARCH, TOKYO (JP); CHUGAI SEIYAKU KABUSHIKI KAISHA, TOKYO (JP)

(21) Appl. No.: 10/543,078

(22) PCT Filed: Jan. 23, 2004
(86) PCT No.: PCT/JP04/00605

§ 371 Date: Jul. 21, 2005

(30) Foreign Application Priority Data
Jan. 24, 2003 (JP) 2003-016750

(51) Int. Cl. A61K 38/16; A61K 48/00; C07H 21/02; C12Q 1/70; C12N 15/86; C07K 14/18

(52) U.S. Cl. 514/12; 514/44; 435/456; 530/350; 536/23.72; 435/5

(57) ABSTRACT

A method of inhibiting the replication ability of a hepatitis C virus (HCV) is provided. An oligoribonucleotide or a peptide nucleic acid which sequence-specifically binds to the HCV-RNA, and a therapeutic agent for hepatitis C which contains any of these components as an active ingredient are provided.

### TECHNICAL FIELD

The present invention relates to an oligoribonucleotide or a peptide nucleic acid which inhibits action of a hepatitis C virus, a vector which expresses the oligonucleotide, a therapeutic agent for hepatitis C which contains any of these components as an active ingredient and a method of inhibiting the replication ability of a hepatitis C virus by allowing the oligoribonucleotide or peptide nucleic acid to bind to RNA of a hepatitis C virus.

### BACKGROUND ART

Hepatitis C virus (hereinafter referred to as “HCV”) is a virus which is a main cause of non-A non-B hepatitis after transfusion, and the cDNA of a gene thereof was cloned in 1989. Many researches have been conducted on HCV using a cloned gene cDNA until now, and particularly socially important achievements such as infection prevention and establishment of a diagnostic method have been resulted, leading to a situation at present where HCV infection after transfusion is scarcely observed. However, the number of HCV carriers in the world is estimated to reach several % of all the population.

It is known that the hepatitis caused by HCV infection has a feature of becoming chronic over a long period of time, resulting in chronic hepatitis, which may lead to liver cirrhosis and further to hepatic cancer at a very high rate, and therefore, a reliable remedy for hepatitis after HCV infection is an important subject.

Although interferon (IFN) therapy is widely performed for treating chronic hepatitis C, there exist problems that the rate of effect is about 30%, that side effects such as fever are induced highly frequently, and that the drug is expensive. IFN has been examined by type, dosage or administration and its effectiveness is expected to be improved by the development of consensus IFN etc., and treatment by a combined use of IFN and an antiviral agent such as ribavirin has been attempted, but none of them has resulted in a reliable remedy so far.

In the meantime, to suppress expression of a specific gene within an animal cell in a living body, a method of suppressing expression of the target gene using a double-stranded RNA corresponding to the target gene has been found recently (Fire A et al., 1998, Nature, vol. 391, p. 806-811). This method is called RNA interference (RNAi) and refers to a phenomenon that when a double-stranded RNA (dsRNA) is introduced in a cell, mRNA in the cell corresponding to the RNA sequence is specifically decomposed, and the protein encoded by the mRNA is no longer expressed. RNAi is an effective method for investigating the function of a novel gene by preventing gene expression, and is extensively used for functional gene analysis in nematode, drosophila, etc.

However, whether RNAi is effective in the treatment of diseases, particularly viral diseases such as hepatitis C has been unknown.

### DISCLOSURE OF THE INVENTION

The present inventors have conducted intensive studies and consequently found that oligoribonucleotides (hereinafter also referred to as “oligo RNAs”) or peptide nucleic acids which sequence-specifically bind to the RNA of HCV (HCV-RNA) inhibit HCV replication, and thus completed the present invention.

That is, the present invention provides the following (1) to (13):

(1) An oligoribonucleotide or peptide nucleic acid which sequence-specifically binds to the RNA (HCV-RNA) of a hepatitis C virus (HCV).

(2) The oligoribonucleotide or peptide nucleic acid according to the above (1) which hybridizes with the RNA of HCV under stringent conditions.

(3) The oligoribonucleotide or peptide nucleic acid according to the above (1) characterized in that the oligoribonucleotide or peptide nucleic acid hybridizes with the sequence of a 5′ non-coding region of the RNA of HCV.

(4) The oligoribonucleotide or peptide nucleic acid according to the above (1) characterized in that the oligoribonucleotide or peptide nucleic acid hybridizes with the sequence of a highly identical region of the genetic sequences of a plurality of types of HCV different in genotype.

(5) The oligoribonucleotide or peptide nucleic acid according to the above (1) which is a double-stranded RNA.

(6) The oligoribonucleotide or peptide nucleic acid according to the above (1) which has a chain length of 19 to 23 bp.

(7) An oligoribonucleotide having a nucleotide sequence shown in any one of SEQ ID Nos. 20 to 34.

(8) An oligoribonucleotide which hybridizes under stringent conditions either with an RNA region of HCV having a sequence complementary to the oligoribonucleotide according to above (7) or an RNA region of HCV hybridizing under stringent conditions with said oligoribonucleotide.

(9) An oligoribonucleotide represented by a nucleotide sequence consisting of 19 to 23 contiguous bases in any one of the nucleotide sequences shown in SEQ ID Nos. 47 to 55.

(10) An oligoribonucleotide which hybridizes under stringent conditions either with an RNA region of HCV having a sequence complementary to the oligoribonucleotide according to above (9) or an RNA region of HCV hybridizing under stringent conditions with said oligoribonucleotide.

(11) A vector which expresses the oligoribonucleotide according to any one of above (1) to (10).

(12) A therapeutic agent for hepatitis C containing as an active ingredient the oligoribonucleotide or peptide nucleic acid according to any one of above (1) to (10) or the vector according to above (11).

(13) A method of inhibiting replication ability of HCV by allowing the oligoribonucleotide or peptide nucleic acid according to any one of above (1) to (10) to bind to the HCV-RNA.

The contents described in the specification and/or drawings of the Japanese Patent Application No. 2003-016750 from which the priority is claimed in the present application are incorporated in the present specification.

### BRIEF DESCRIPTION OF THE DRAWINGS

FIG. 1 shows a general secondary structure in the 5′ non-coding region of the HCV-RNA;

FIG. 2 shows a general secondary structure in the 3′ non-coding region of the HCV-RNA;

FIG. 3A shows cDNA sequences of 500 bases from the 5′ non-coding region to the core region of the RNAs of HCV-1, HCV-BK and HCV-J which are isolated strains of HCV;

FIG. 3B shows cDNA sequences of 500 bases from the 5′ non-coding region to the core region of the RNAs of R6, R24 and S14J which are isolated strains of HCV;

FIG. 3C shows cDNA sequences of 500 bases from the 5′ non-coding region to the core region of the RNAs of HCJ6, JFH1 and JCH1 which are isolated strains of HCV;

FIG. 3D shows cDNA sequences of 500 bases from the 5′ non-coding region to the core region of the RNAs of JCH3 and HCJ8 which are isolated strains of HCV;

FIG. 4A shows a part of the results by multiple alignment of 500 bases from the 5′ non-coding region to the core region of various types of HCV;

FIG. 4B shows the results (sequel to FIG. 4A) by multiple alignment of 500 bases from the 5′ non-coding region to the core region of various types of HCV;

FIG. 4C shows the results (sequel to FIG. 4B) by multiple alignment of 500 bases from the 5′ non-coding region to the core region of various types of HCV;

FIG. 5A shows cDNA sequences for the RNA of the 3′ non-coding region of pH77J6S, R6, R24L and R24S;

FIG. 5B shows cDNA sequences for the RNA of the 3′ non-coding region of HCJ6CH, JFH1, JCH1 and 2 b_AB030907;

FIG. 6 shows a relation between the addition of siRNA and the amount of HCV core protein produced by Rz-HepM6 cell line;

FIG. 7 shows the relation between the addition of siRNA and the activity by which HCV replicon is replicated;

FIG. 8 shows the relation between the addition of siRNA prepared by dicer processing and the activity which HCV replicon replicates; and

FIG. 9 shows the relation between the addition of siRNA prepared by dicer processing and the activity which HCV replicon replicates.

### BEST MODE FOR CARRYING OUT THE INVENTION

The oligo RNA of the present invention which sequence-specifically binds to the HCV-RNA is an oligonucleotide having ribose as sugar and encompasses those containing as a base adenine, guanine, cytosine and uracil which exist in natural RNA, as well as thymine and other modified bases, etc. Although the oligo RNA of the present invention is not particularly limited as far as the oligo RNA can sequence-specifically bind to the HCV-RNA, it is preferable that it is an oligo RNA which inhibits the replication ability of HCV. Examples of the oligo RNA which can sequence-specifically bind to the HCV-RNA include an oligo RNA having a sequence complementary to a sequence of the HCV-RNA, an oligo RNA having a sequence exhibiting a high identity to a sequence complementary to a sequence of the HCV-RNA and an oligo RNA which can hybridize with RNA having a sequence of the HCV-RNA under stringent conditions. In addition, although the present invention is not restrained by any specific theory, it is considered that siRNA, which is one preferable embodiment of the present invention, hybridizes with a target gene within a cell and cleaves the target gene via dicer, and that the target gene is cleaved to the length of 19 to 23 nt. On the other hand, an antisense nucleic acid, which is another embodiment of the present invention, hybridizes with a target gene to induce IFN and activate RNase, thereby decomposing the target gene. Alternatively it is supposed that it binds to the target gene to cause structural change of the target RNA and inhibits translation. Although the sequence of the HCV-RNA includes either of the genomic RNA sequence (− strand) of HCV or the mRNA sequence (+ strand) transcribed from the genomic RNA in the present invention, +strand sequence is more preferred.

In addition, siRNA means an oligo RNA having a length of 19 to 23 nt (19 to 23 bp) in this specification. When the siRNA forms a double strand, one or both of them may have a protruding end.

In the present invention, high identity means identity of 70% or more, preferably identity of 80% or more, and more preferably identity of 90% or more (for example, identity of 95% or more). The algorithm BLAST by Karlin and Altschul (Proc. Natl. Acad. Sci. USA 90:5873-5877, 1993) etc. can be used to determine the identity of base sequences. The programs called BLASTN and BLASTX have been developed based on this algorithm (Altschul et al. J. Mol. Biol. 215:403-410, 1990). When base sequences are analyzed by BLASTN based on BLAST, parameters are set, for example to score=100 and wordlength=12. When amino acid sequences are analyzed by BLASTX based on BLAST, parameters are set, for example to score=50 and wordlength=3. When BLAST and Gapped BLAST programs are used, default parameters of each program may be used. Specific technique of these analytical methods is well-known (http://www.ncbi.nlm.nih.gov.).

The hybridization technique is a technique well known in the art (for example, Sambrook, J et al., Molecular Cloning 2nd ed., 9.47-9.58, Cold Spring Harbor Lab. press, 1989, etc.) and those skilled in the art can select suitable stringent conditions. Examples of stringent conditions include for example, conditions of 42° C., 5×SSC and 0.1% SDS in the washing after hybridization, preferably conditions of 50° C., 5×SSC and 0.1% SDS, and more preferably conditions of 65° C., 0.1×SSC and 0.1% SDS. However, as for factors which influence the stringency of hybridization, plural factors such as temperature and salt concentration can be considered, and those skilled in the art could select and adjust these factors suitably to achieve the similar stringency.

The oligo RNA of the present invention may be single stranded or double stranded, or may be multiple stranded formed of two or more strands but preferably double stranded. The double strand may be formed of two independent strands or may be a double stranded structure formed in a self-complementary single stranded RNA, which can form a stem-loop structure by one molecule. When an oligo RNA is double stranded, it may be double stranded in all regions or have some regions (for example, both ends or one end, etc.) of other structures such as single strand.

The length of the oligo RNA of the present invention is not limited as long as it has a sequence-specific binding ability to the HCV-RNA. Examples of the length of the oligo RNA of the present invention include 5 to 1000 bases (5 to 1000 bp in the case of double strand), preferably 10 to 100 bases (10 to 100 bp in the case of double strand), more preferably 15 to 25 bases (15 to 25 bp in the case of double strand), and particularly preferably 19 to 23 bases (19 to 23 bp in the case of double strand).

In the present invention, a preferable oligo RNA is an oligo RNA which has a nucleotide sequence shown in SEQ ID Nos. 20 to 34, and particularly preferably an oligo RNA which has a nucleotide sequence shown in SEQ ID Nos. 20 to 25. In addition, the other preferable oligo RNAs in the present invention include oligo RNAs represented by the nucleotide sequence which consists of 19 to 23 contiguous bases in the nucleotide sequence shown in SEQ ID Nos. 47 to 55.

Examples of the still other preferable oligo RNAs in the present invention include an oligo RNA which hybridizes under stringent conditions either with an RNA region of HCV having a sequence complementary to the above oligo RNA having a nucleotide sequence shown in SEQ ID Nos. 20 to 34, or with an RNA region of HCV hybridizing under stringent conditions with said oligo RNA, and an oligo RNA which hybridizes under stringent conditions either with an RNA region of HCV represented by a nucleotide sequence consisting of 19 to 23 contiguous bases in nucleotide sequences shown in SEQ ID Nos. 47 to 55, or with an RNA region of HCV hybridizing under stringent conditions with said oligoribonucleotide. Those skilled in the art could readily determine in any type of HCV an RNA region of HCV which hybridizes under stringent conditions with these RNAs. Examples of these oligo RNAs include a nucleotide sequence described in SEQ ID Nos. 20 to 34 or a nucleotide sequence consisting of 19 to 23 contiguous bases in nucleotide sequences shown in SEQ ID Nos. 47 to 55 wherein 7 or less, preferably 5 or less, more preferably 3 or less nucleotides are deleted, substituted or added and which can inhibit HCV replication by hybridizing with the RNA of HCV.

In addition, peptide nucleic acids which can be suitably used in the present invention include peptide nucleic acids having base sequences corresponding to oligo RNAs which can be suitably used in the present invention.

The RNA of HCV consists of a non-coding region on the 5′-end side (5′-end non-coding region) containing about 340 nucleotides, an open reading frame (ORF) of about 9,400 nucleotides, and a non-coding region on the 3′-end side (3′-end non-coding region) containing about 50 nucleotides. Although a site targeted by the oligo RNA of the present invention is not particularly limited but may be any site of the RNA sequence, the target site is preferably located from the 5′ non-coding region to the 5′-end region of ORF (for example, regions having base sequences shown in SEQ ID Nos. 1 to 11) or in the 3′ non-coding region (for example, regions having base sequences shown in SEQ ID Nos. 12 to 19), more preferably the 5′-end non-coding region.

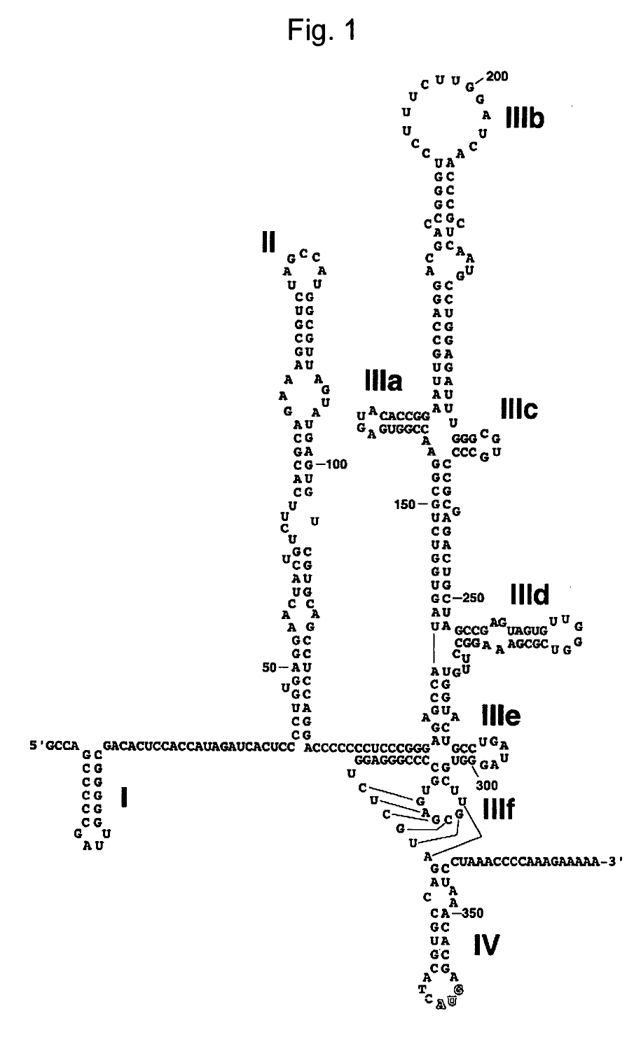

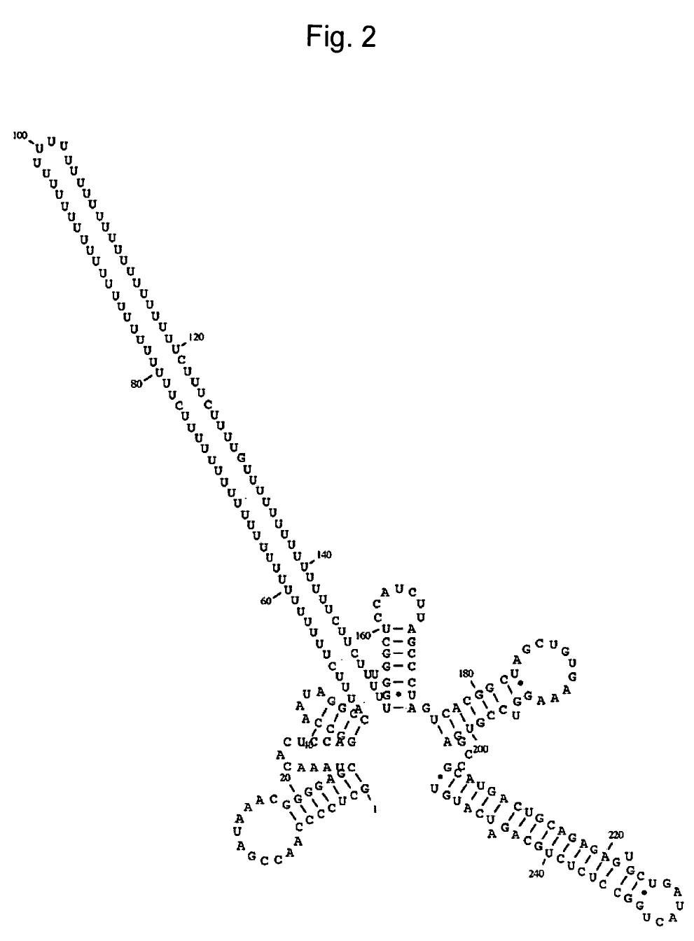

An internal ribosomal entry site (IRES) and stem regions which form a stem loop are present in the 5′ non-coding region of the HCV-RNA. There have been many reports on the 5′-end non-coding region, IRES and the stem region for HCV (Kato. N. et al., Proc. Natl. Acad. Sci. USA., 87, 9524-9528 (1990), Proc. Natl. Acad. Sci. USA., 88, 2451-2455 (1991), J. Viol., 65, 1105-1113 (1991), J. Gen. Viol., 72, 2697-2704 (1991), Virology, 188, 331-341 (1992), Tsukiyama. Kohara et al., J. Virol., 66, 1476-1483 (1992), Honda Masao et al., J. Virol., 73, 1165-1174 (1999), Honda Masao et al., RNA, 2 (10), 955-968 (1996), Sasano T. et al., Genome Inf. Ser., 9, 395-396 (1998), Ito T et al., J. Virol., 72, 8789-8796 (1998), Kamoshita N et al., Virology., 233, 9-18 (1997), etc. General secondary structures in the 5 non-coding region and 3′ non-coding region of the HCV-RNA are shown in FIGS. 1 and 2.

As for HCV, there are plural types of HCV which are different from each other in genotype. Examples thereof include HCJ6, HCJ8, HCV-1, HCV-BK, HCV-J, JCH1, JCH3, JFH1, R24, R6, S14J, pH77J6S (GenBank Accession no. AF177039), HCJ6CH, 2 b_AB030907, etc. In order to deal with these plural HCV-RNAs which are different from each other in genotype, it is preferable to target a region having a high identity among the plural HCV gene sequences which are different from each other in genotype. The region having a high identity among the plural HCV gene sequences which are different from each other in genotype as used herein means a region where the RNA sequences of plural types of HCV have an identity of 80% or more, preferably an identity of 90% or more, more preferably an identity of 95% or more. Such a region preferably has a length of 10 or more bases, more preferably a length of 15 or more bases, and particularly preferably a length of 20 or more bases. Plural types of HCV as used herein usually refer to 3 or more types of HCV, preferably 5 or types of HCV and particularly preferably 10 types of HCV. The identity of the gene sequences can be calculated by comparing of the plural types of the object genes and applying the above-mentioned algorithm and the like.

There are no particular limitation on the oligoribonucleotide used in the present invention, and in addition to those having normal RNA constructs which are not modified, modified RNAs having a phosphate diester moiety or a sugar moiety modified can be used. Moreover, the oligo RNA of the present invention may contain as its portion a non-ribonucleotide molecule such as deoxyribonucleotide.

In addition, a peptide nucleic acid (PNA) etc. can be used instead of an oligo RNA in the present invention. PNA is a technique well known in the art (Nielsen Peter E., Methods in Molecular Biology, 208, 3-26 (2002); Braasch Dwaine A et al., Biochemistry, 41 (14), 4503-4510 (2002); Koppelhus Uffe et al., Antisense Drug Technology, 359-374 (2001); Nielsen Peter E., Methods in Enzymology, 340, 329-340 (2001)) and like the above-mentioned oligo RNA, a PNA which can sequence-specifically bind to the HCV-RNA can be prepared. The length of a suitable peptide nucleic acid in the present invention is, for example, 5 to 1,000 bases (5 to 1,000 bp in the case of double strand), preferably 10 to 100 bases (10 to 100 bp in the case of double strand), more preferably 15 to 25 bases (15 to 25 bp in the case of double strand) and particularly preferably 19 to 23 bases (19 to 23 bp in the case of double strand).

The oligo RNA or peptide nucleic acid of the present invention can be prepared by well-known methods to those skilled in the art.

When the oligo RNA of the present invention is to be expressed continuously, a vector which expresses the oligo RNA of the present invention may be prepared. A vector can be prepared by well-known methods to those skilled in the art. For example, it can be prepared by introducing a gene encoding the oligo RNA of the present invention into a well-known vector such as those described in Nature Biotech (2002) 19, 497-500. Promoters suitable for the expression of the oligo RNA of the present invention are not particularly limited, and examples thereof include T7 promoter, tRNA promoter, U6 promoter, etc.

Since the oligo RNA of the present invention can inhibit HCV replication and suppress HCV multiplication, it is useful as a therapeutic agent for hepatitis C. In this case, when an oligoribonucleotide or peptide nucleic acid corresponding to plural types of HCV is provided, treatment can be effected without clinically identifying the type of a virus with which the patient is infected and it is not necessary to use a combination of plural types of oligoribonucleotide or peptide nucleic acid and therefore, such an embodiment is preferable.

When the drug is used for medical treatment, it can also be administered in the form which can function within a cell as it is. In this case, about 19 to 23 bases are optimal as the length of the oligo RNA or peptide nucleic acid. Moreover, the drug can also be administered in the form which can function through processing in a cell. In this case, the oligo RNA or peptide nucleic acid which has a longer sequence than the sequence including the desired sequence can be administered. The double-stranded RNA (dsRNA) taken into the cell is decomposed to about 21mer by an enzyme called dicer, and serves as siRNA (short-interfering RNA), and forms a complex called RISC(RNA-Induced Silencing Complex) which is supposed to destroy RNA having a specific base sequence transcribed from the genome (Bernstein, E. et al., Nature, 409:363-366, 2001; Hammond, S. M. et al., Nature, 404:293-296, 2000). Alternatively, siRNA prepared beforehand in vitro using a commercially available dicer can also be used.

The therapeutic agent for hepatitis C comprising the oligo RNA or the peptide nucleic acid of the present invention as an active ingredient can be prepared by adding pharmaceutically acceptable excipients, tonicity agent, dissolution auxiliary agent, stabilizing agent, antiseptic, soothing agent, etc. if needed, to form a pharmaceutical composition such as a tablet, powder, granule, capsule, liposome capsule, injection, liquid and nasal drop, and can be further a lyophilized agent. These can be prepared according to usual methods. In addition, it is also possible to administer a vector which expresses the oligo RNA of the present invention.

Although the administration route of the oligo RNA or peptide nucleic acid of the present invention is not particularly limited, it is applied to a patient so that it may finally reach the affected site, preferably for example, by directly applying it to the affected site of the patient or administering it into a blood vessel. Furthermore, an enclosure material which enhances durability and membrane permeability can also be used. Examples thereof include liposome, poly-L-lysine, lipid, cholesterol, lipofectin and a derivative thereof.

The dosage of the oligo RNA or peptide nucleic acid of the present invention can be suitably adjusted according to the patient's condition to provide a preferable amount. For example, it can be administered in a range of 0.001 to 100 mg/kg, preferably 0.1 to 10 mg/kg, but it is not particularly limited.

Furthermore, the present invention provides a method of inhibiting the replication ability of HCV by allowing the above-mentioned oligoribonucleotide or peptide nucleic acid of the present invention to bind to the RNA of HCV. The method of the present invention includes contacting a sample, which contains or may contain HCV, with an oligo RNA or peptide nucleic acid of the present invention either in vivo or in vitro. The result of prevention of the replication ability of HCV can be detected by methods usually used in the art.

The present invention is described further referring to Examples hereinbelow, but the present invention is not limited to these Examples.

### EXAMPLE 1

### Determination of the Region to be Targeted by siRNA

Sequences were compared in order to find regions having a high homology in the base sequence which constitutes the gene in many isolated strains of hepatitis C virus, particularly from 5′ non-coding region to the core region and in 3′-end non-coding region.

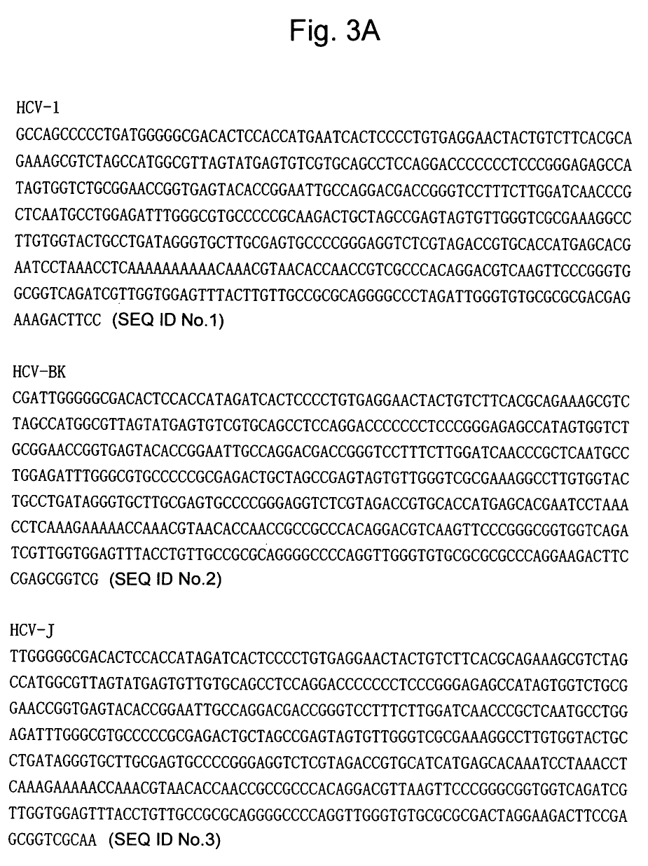

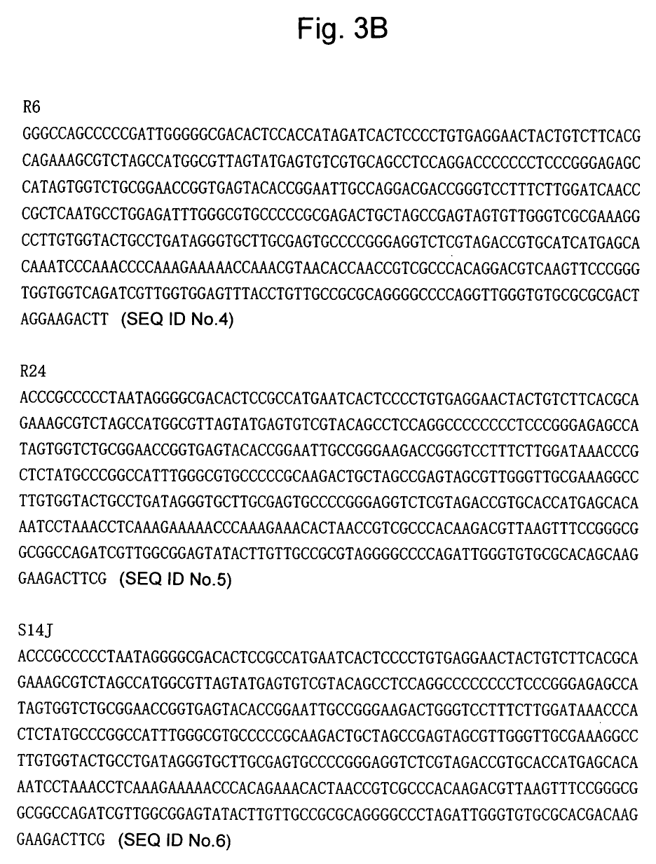

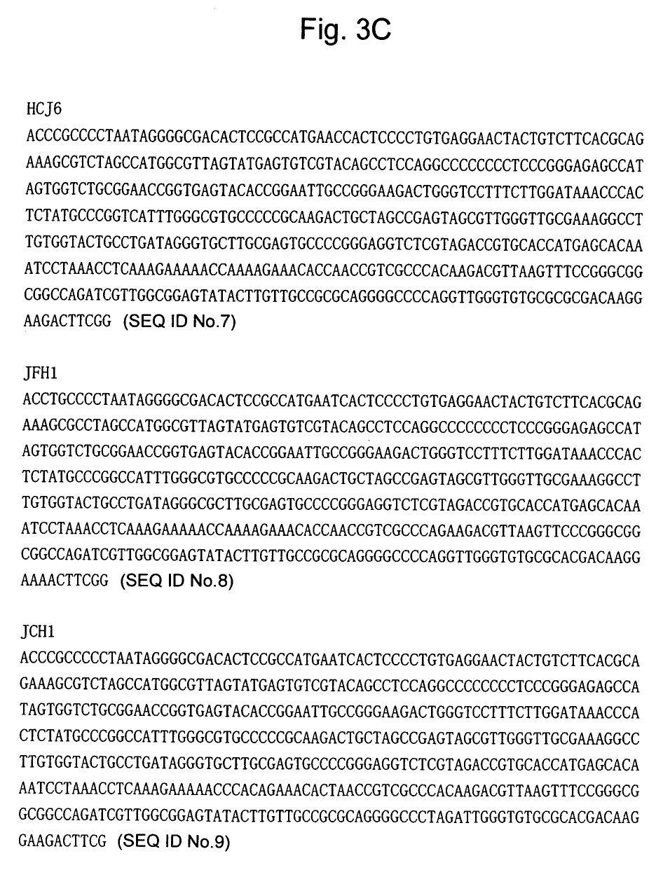

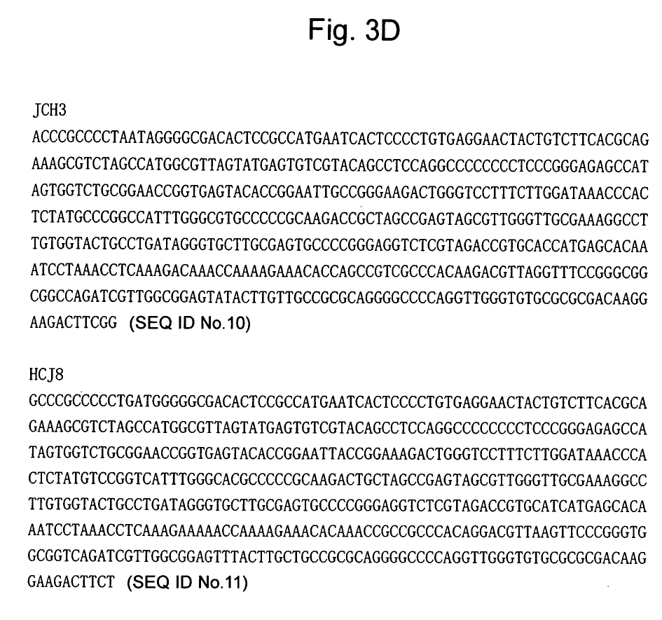

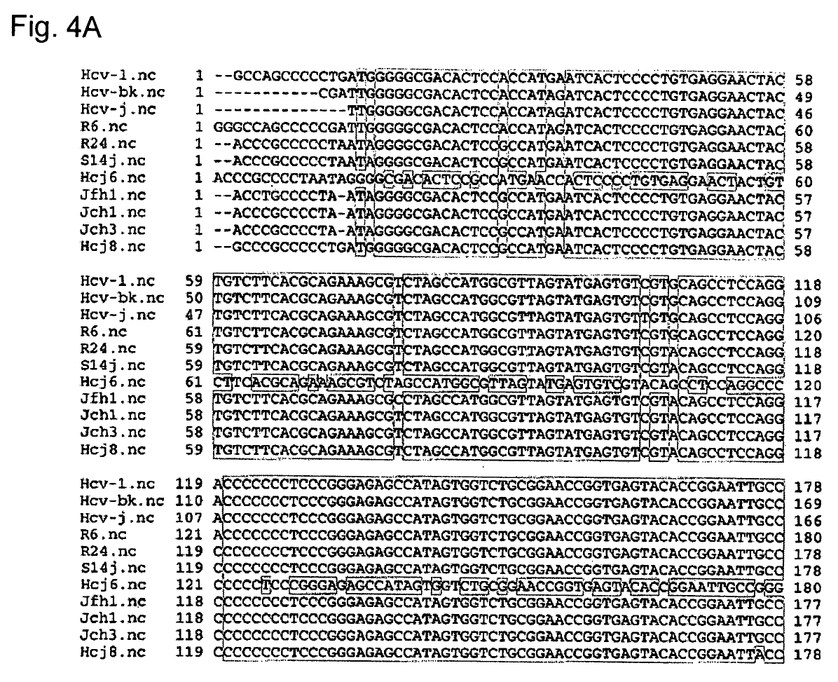

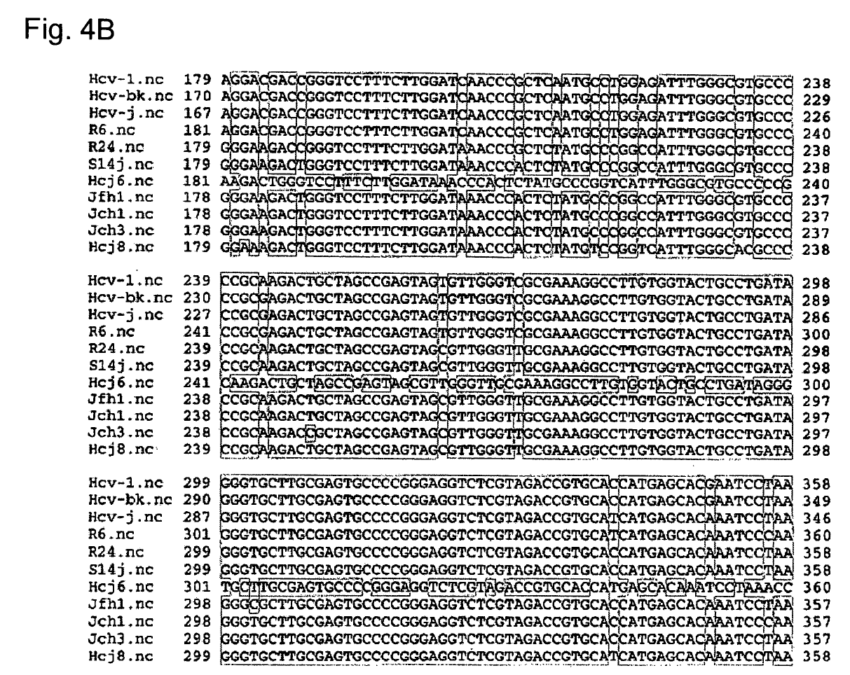

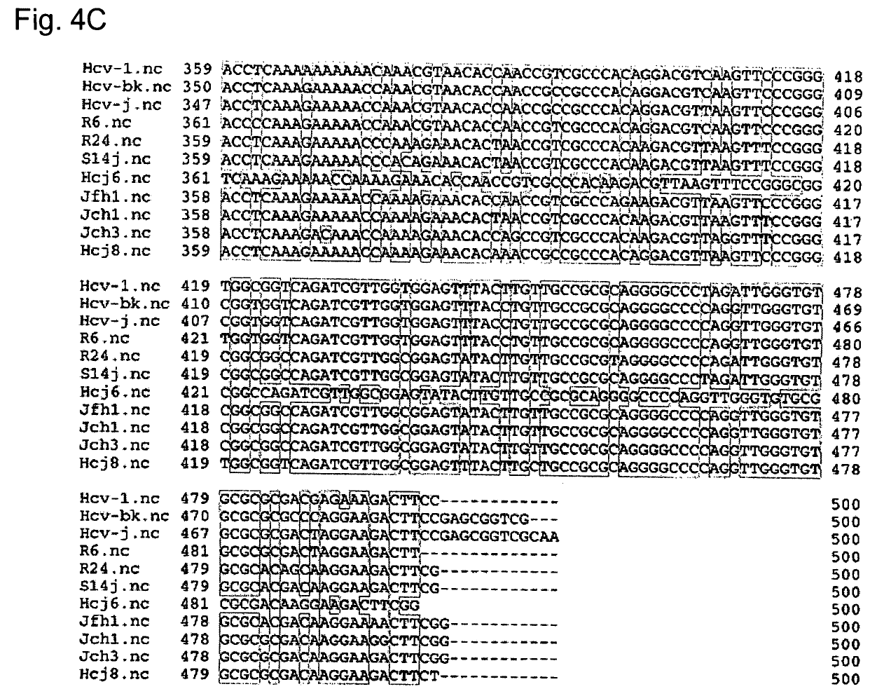

The cDNA sequences of about 500 bases from 5′ non-coding region to the core region of the RNA of HCV-1 (GenBank Accession no. M62321), HCV-BK (Accession no. M58335), HCV-J (Accession no. D90208), R6 (Accession no. AY045702), R24, S14J, HCJ6, JFH1 (Accession no. AB047639), JCH1 (Accession no. AB047640), JCH3 (Accession no. AB047642), HCJ8 (Accession no. D10988) which are isolated strains of HCV are shown in FIG. 3. Multiple alignment was performed using the method usually performed in the art for these base sequences. The results are shown in FIG. 4. Based on the region on the 5′-end side of HCV in which at least 10 types of HCV have an identity of 95% or more, siRNA which can sequence-specifically bind to this region as the target was designed.

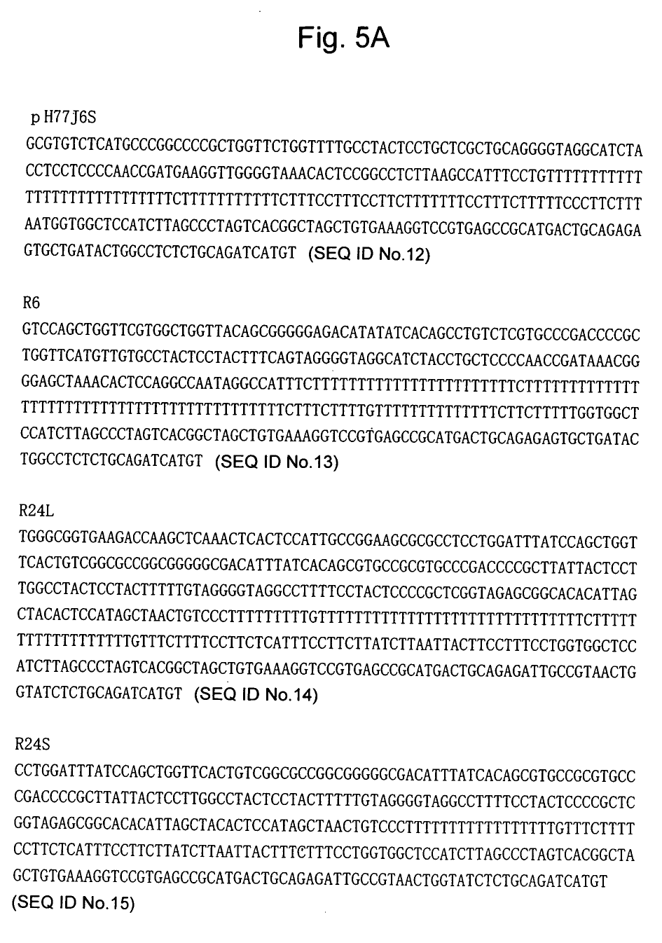

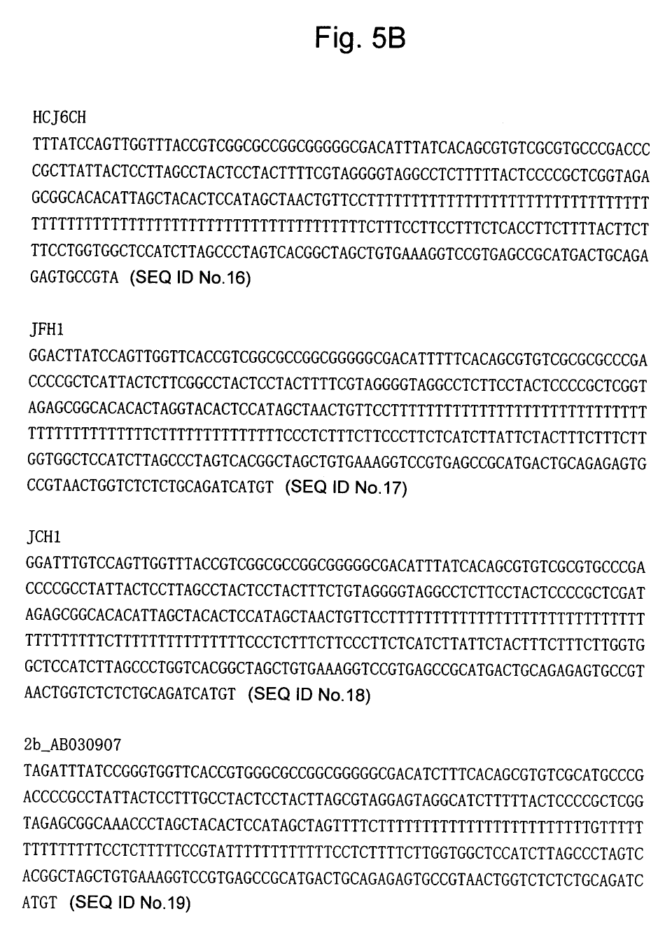

Multiple alignment was similarly carried out for the non-coding region on the 3′-end side using the sequences of pH77J6S (Accession no. AF177039), R6, R24L, R24S, HCJ6CH (Accession no. AF177036), JFH1, JCH1 and 2 b_AB030907 (Accession no. AB030907) which were similarly shown in FIG. 5, and based on the region in which at least 8 types of HCV have an identity of 95% or more, siRNA which can sequence-specifically bind to this region as the target was designed.

In the meantime, it is preferable to design siRNA in consideration of the above-mentioned sequence identity, and in consideration that it may be bound to the region just before the translation initiation site. Furthermore, it is preferable to design also in consideration of the secondary structure of HCV RNA which serves as the target. Particularly, 5′- and 3′-UTR loop structure and their neighboring regions can be used as a target.

### EXAMPLE 2

### Synthesis of siRNA

Based on the results of Example 1, a sequence of siRNA having a length of 21 nt was designed to all the target HCV genomes, and an oligonucleotide which includes T7 promoter sequence in the 3′-end was synthesized according to the protocol of Silencer siRNA Construction Kit (Ambion cat. no. 1620). 100 μM of each oligonucleotide used as a template was prepared, allowed to hybridize with T7 primer, and converted to a double-stranded DNA using Klenow enzyme and transcribed using T7 promoter. After carrying out annealing of the synthesized RNA to each complementary strands to form a double-stranded RNA, the remaining single stranded protruding ends were digested by RNase to prepare a siRNA. 15 to 30 μg/reaction of siRNA finally synthesized was adjusted to 10 μM with a RNase free water, and after the presence of the double-stranded RNA of 20 to 22 bases was confirmed by 12% acrylamide gel electrophoresis, it was stored at −80° C. until it was used.

The synthesized siRNA sequences were shown below. The nucleotide numbers in the sequence of HCV (R6 strain) (Accession no. AY045702, SEQ ID No. 56) to which these sequences correspond are also indicated.

1) siRNA Targeting 5′-UTR

| | | | |
|---|---|---|---|
| R1-siRNA; | 5′-GGAACUACUGUCUUCACGCAG-3′ | (21 bases) | (SEQ ID No. 20, 53-73 nt) |
| R2-siRNA; | 5′-GCCAUAGUGGUCUGCGGAACC-3′ | (21 bases) | (SEQ ID No. 21, 139-159 nt) |
| R3-siRNA; | 5′-AGGCCUUGUGGUACUGCCUGAU-3′ | (22 bases) | (SEQ ID No. 22, 278-299 nt) |
| R5-siRNA; | 5′-GUCUCGUAGACCGUGCAUCA-3′ | (20 bases) | (SEQ ID No. 23, 325-344 nt) |
| R6-siRNA; | 5′-GCGAAAGGCCTTGTGGTACTG-3′ | (21 bases) | (SEQ ID No. 24, 273-293 nt) |
| R7-siRNA; | 5′-GTCTCGTAGACCGTGCACCA-3′ | (20 bases) | (SEQ ID No. 25, 325-344 nt) |
| R5L-siRNA; | 5′-GUCUCGUAGACCGUGCAUCAT-3′ | (21 bases) | (SEQ ID No. 26, 325-345 nt) |
| R1mut-siRNA; | 5′-GGAACUACUGUCUUCACGCAG-3′ | (21 bases) | (SEQ ID No. 27, 53-73 nt) |
| R2mut-siRNA; | 5′-GCCAUAGUGGUCUGCGGAACC-3′ | (21 bases) | (SEQ ID No. 28, 139-159 nt) |
| R3mut-siRNA; | 5′-AGGCCUUGUGGUACUGCCUGAU-3′ | (22 bases) | (SEQ ID No. 29, 278-299 nt) |
| R5mut-siRNA; | 5′-GUCUCGUAGACCGUGCAUCA-3′ | (20 bases) | (SEQ ID No. 30, 325-344 nt) |
| R6mut-siRNA; | 5′-GCGAAAGGCCTTGTGGTACTG-3′ | (21 bases) | (SEQ ID No. 31, 273-293 nt) |
| R7mut-siRNA; | 5′-GTCTCGTAGACCGTGCACCA-3′ | (20 bases) | (SEQ ID No. 32, 325-344 nt) |

2) siRNA Targeting 3′-UTR

| | | | |
|---|---|---|---|
| R8-siRNA; | 5′-GGCTCCATCTTAGCCCTAGTC-3′ | (21 bases) | (SEQ ID No. 33, 9515-9535 nt) |
| R9-siRNA; | 5′-GGCTAGCTGTGAAAGGTCCGT-3′ | (21 bases) | (SEQ ID No. 34, 9538-9558 nt) |

### EXAMPLE 3

### Effect on the Expression of the Core Protein of HCV

The present inventors have already established a system in which switching expression of all the HCV genomes are carried out by Cre/loxP system (J. Biol. Chem., 273, 9001-6. (1998)). The present inventors have established a human origin liver cell line Rz-HepM6 which carries out self-sustaining expression of all the HCV genomes (Genotype Ib, nucleotide no. 1-9611nt) using Cre recombinase this time, and this was used as the target. The Rz-HepM6 cell was suspended to Dulbecco's Modified Eagle medium (NISSUI cat. no. 05915) which contains 10% fetal bovine serum (REHATUIN cat. no. 1020-90), and seeded on a 24-well plate in 10⁵ cells per well, and cultured overnight at 37° C. under 5% CO₂. siRNA was introduced when the cell density was 50 to 70%. That is, 2.0 μl of Oligofectamine transfection reagent (Invitrogen cat. No. 12252-011) and 5.5 μl of Opti-MEMI (Gibco cat. No. 22600) was added and mixed well, and it was allowed to stand still for 10 minutes at room temperature. Then, 5.0 μl of synthesized 10 μM siRNA was diluted in 40 μl of Opti-MEMI and added so that the final concentration might be 200 nM. After allowed to stand still for 20 minutes at room temperature, siRNA to which the Oligofectamine reagent was added was directly added to the cell for which the culture solution was exchanged to 200 μl of Opti-MEMI beforehand and cultured at 37° C. under 5% CO₂.

In 4 hours after siRNA was added, 125 μl of Dulbecco's Modified Eagle medium containing 30% fetal bovine serum, which was three times in the concentration, was added, and cultured at 37° C. under 5% CO₂. Cells were collected in 24 hours after the serum was added with 20 μl of lysis buffer (1% SDS, 0.5% NP40, 0.15M NaCl, 0.5 mM EDTA, 1 mM DTT, 10 mM Tris:pH7.4), and quantification of the HCV core protein was carried out using a HCV core quantification kit (International Reagents cat. No. 14861).

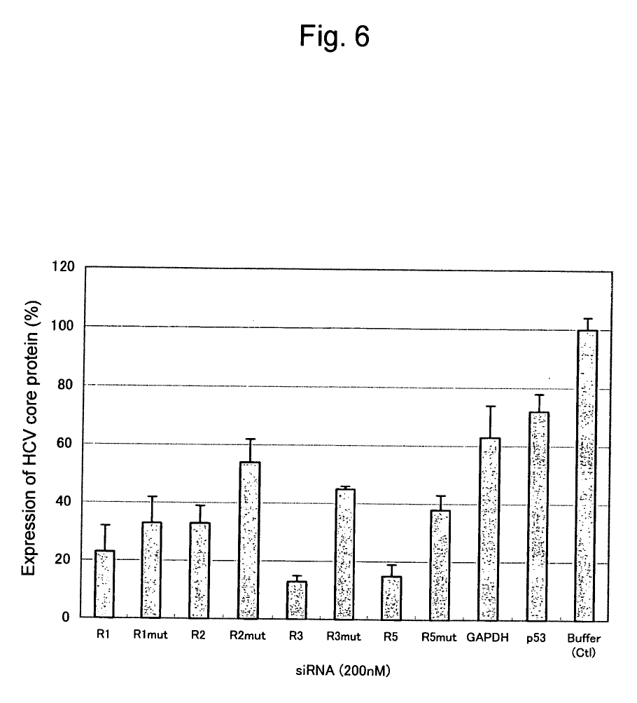

The relation between the addition of siRNA and the amount of HCV core protein which Rz-HepM6 cell line produces is shown in FIG. 6. The quantification of the core protein which constitutes a virus particle was carried out by ELISA method after adding 200 μM each of siRNA (R1-siRNA, R2-siRNA, R3-siRNA, R5-siRNA, Rlmut-siRNA, R2mut-siRNA. R3mut-siRNA and R5mut-siRNA). Although all of the added siRNAs inhibited synthesis of the core protein, it was observed that particularly the action of R3 and R5 was strong, and the specificity of their base sequences was also high. Furthermore, inhibitory effect of the expression of the core protein by R3mut and R5mut, sequences into which variation was introduced, was decreased.

### EXAMPLE 4

### Replicon Assay

In order to carry out the quantification of the number of copies of the HCV-RNA, those having a luciferase gene derived from firefly as a reporter gene introduced into the HCV-RNA were constructed. According to the method by Krieger et al. (J. Virol., 75, 4614-24 (2001)), the luciferase gene was introduced immediately after the Internal Ribosome Entry Site (IRES) of HCV gene in the form a fused gene with neomycin resistance gene. After the RNA of interest was synthesized in vitro, it was introduced into Huh7 cell (Japanese Collection of Research Bioresources) by electroporation method, and isolated as a G418 resistance clone. The firefly luciferase HCV replicon cell (Huh-3-1) was suspended to Dulbecco MEM (Gibco cat. no. 10569) which contains 5% fetal bovine serum (Hyclone cat. no. SH 30071.03), and 5000 cells per well were seeded on a 96-well plate and cultured overnight at 37° C. under 5% CO₂. In about 20 hours, diluted siRNA was added in an amount of 10 μl per well, and was cultured for three more days. The assay plate was prepared in two lines, one assay was performed on the white plate and the other was performed on the clear plate.

The white plate was used for Steady-Glo Luciferase Assay System (Promega cat. no. E2520) after the culturing was finished. That is, 100 μl of the reagent per well was put in, mixed with a pipette 3 to 4 times, and luminescence was measured by 1450 MicroBeta TRILUX (WALLAC) after allowing it to stand still for 5 minutes.

The synthesized siRNAs were introduced into the HCV replicon cells by the following methods. That is, 10000 cells per well were seeded on a 96-well plate and cultured overnight at 37° C. under 5% CO₂. siRNA was introduced when the cell density was 50 to 70%. That is, 1.5 μl of TransIT-TKO transfection reagent (Mirus Corporation cat. No. MIR2150) and 25 μl of Opti-MEMI (cata no. 31985) were vigorously agitated and then allowed to stand still for 20 minutes. 0.125 to 1.25 μl of siRNA was mixed and they were gently stirred and allowed to stand still for further 20 minutes. This solution was calmly added to 100 μl of the cell in 96-well plate, and cultured overnight at 37° C. under 5% CO₂. Replicon assay was performed using these cells. siRNA was added so that the final concentration might be 1 nM, 10 nM, 30 nM and 100 nM and introduced with TransIT-TKO transfection reagent, and in 24 hours, HCV replicon activity was measured using the reporter gene (luciferase activity) as an index. The activity of each siRNA was calculated by subtracting the value in which the cells were not added as a background from all the values and assuming the activity when no siRNA was added as 100%.

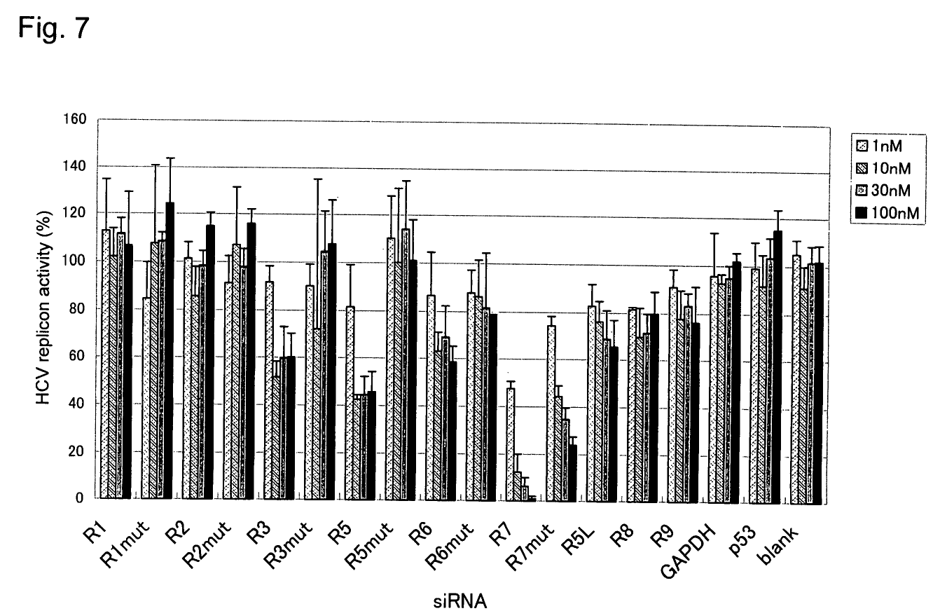

The relation between the addition of siRNA and the activity by which HCV replicon is replicated is shown in FIG. 7. Sequences R3, R5, R6 and R7 inhibited the activity of replicon in dose dependence. Sequence R3mut, R5mut, R6mut and R7mut in which the base sequences are partially substituted have decreased effect and therefore it is considered that the sequences R3, R5, R6 and R7 exhibit sequence-specific antivirotic effect.

### EXAMPLE 5

### Calculation of RNA Transfection Efficiency

siRNA was labeled with Cy3 using Silencer siRNA Labeling Kit (Ambion cat no. 1632) according to the protocol. That is, of 7.5 μl of Cy3 labeling reagent was added to 10 μM (19.2 μl) of R7-siRNA, and the labeling was performed at 37° C. in a shaded condition in 50 μl for 1 hour. 5 μl of 5M NaCl and 99.5% ethanol 2.5 times in volume were added and ethanol precipitation was performed at −20° C. The Cy3-labeled siRNAs were collected by centrifugation at 4° C., 15000 rpm. The quantification of the labeled siRNA was carried out by calculating from the maximum absorption and the molecular extinction coefficient of Cy3 (http://www.ambion.com/techlib/append/base dye.html).

The obtained Cy3-labeled siRNAs were introduced into the cells using the TransIT-TKO transfection reagent, and were observed with fluorescence microscope in 24 hours. After confirming the positions of the cells in the view of phase-contrast microscope at first, the cells dyed with Cy3 were observed with fluorescence microscope. The wavelength used at this time was 510 nm for the exciting wavelength and 550 nm for the absorption wavelength. It became clear that the Cy3-labeled cells were about 90% of the whole cells, which was very high transfection efficiency.

### EXAMPLE 6

### Preparation of si-RNA by Dicer

HCV R6 gene (Accession no. AY045702, SEQ ID No. 56) was used as a template, each of the combinations shown in Tables 1 and 2 as primers and PCR reaction was performed following the normal method. Primers were designed by selecting regions in which homology among plural types of HCV exists or regions important for the replicative function of HCV. After excising and purifying the obtained PCR product from the gel, transcription reaction (20 μl vol×4 hours) was performed using T7 RNA polymerase (for example, MEGAscript T7, Ambion Inc. cat # 1334), RNA was synthesized, and the double-stranded RNAs of having sizes of the object RNAs were confirmed by agarose gel electrophoresis (precursor siRNA-1 to precursor siRNA-9, SEQ ID Nos. 47 to 55). Subsequently, DNaseI is used and after reacting for 15 minutes, LiCl precipitation was carried out, and the product was dissolved in 20 μl of Nuclease free Water, and the amount of RNA was measured by absorption (total about 30 to 60 μg of dsRNA/reaction).

TABLE 1

| Designation of double- | | |
|---|---|---|
| stranded RNA | Primer 1 | Primer 2 |
| Precursor siRNA-1 | Ds5-41-S25 | Ds5-612-R23 |
| Precursor siRNA-2 | Ds5-41-S25 | Ds5-857-R25 |
| Precursor siRNA-3 | Ds3-8864-S25 | Ds3-9537-R25 |
| Precursor siRNA-4 | Ds3-8864-S25 | Ds3-9611-R23 |
| Precursor siRNA-5 | Ds5-41-S25 | Ds5-397-R23 |
| Precursor siRNA-6 | Ds3-9267-S23 | Ds3-9611-R23 |
| Precursor siRNA-7 | Ds5-201-S25 | Ds5-397-R23 |
| Precursor siRNA-8 | Ds5-261-S25 | Ds5-360-R25 |
| Precursor siRNA-9 | Ds5-311-S25 | Ds5-360-R25 |

TABLE 2

| | |
|---|---|
| Primer | Sequence |

| | |
|---|---|
| Ds5-41-S25 | ACTCCCCTGTGAGGAACTACTGTCT |
| | (SEQ ID No. 35) |
| Ds3-8864-S25 | AGGATGATTCTGATGACCCATTTCT |
| | (SEQ ID No. 36) |
| Ds3-9267-S23 | GCGGGGGAGACATATATCACAGC |
| | (SEQ ID No. 37) |
| Ds5-201-S25 | TGGATCAACCCGCTCAATGCCTGGA |
| | (SEQ ID No. 38) |
| Ds5-261-S25 | TAGTGTTGGGTCGCGAAAGGCCTTG |
| | (SEQ ID No. 39) |
| Ds5-311-S25 | GAGTGCCCCGGGAGGTCTCGTAGAC |
| | (SEQ ID No. 40) |
| Ds5-612-R23 | CCCTCGTTGCCATAGAGGGGCCA |
| | (SEQ ID No. 41) |
| Ds5-857-R25 | AACCGGGCAAATTCCCTGTTGCATA |
| | (SEQ ID No. 42) |
| Ds3-9537-R25 | GACTAGGGCTAAGATGGAGCCACCA |
| | (SEQ ID No. 43) |
| Ds3-9611-R23 | ACATGATCTGCAGAGAGGCCAGT |
| | (SEQ ID No. 44) |
| Ds5-397-R23 | GCGGCGGTTGGTGTTACGTTTGG |
| | (SEQ ID No. 45) |
| Ds5-360-R25 | TTAGGATTTGTGCTCATGATGCACG |
| | (SEQ ID No. 46) |

Then, Dicer siRNA Generation kit (Cat # T510001) available from Gene Therapy Systems. Inc. was used and 10 to 20 μg each of dsRNA was reacted with 10 unit (20 μl) of the dicer protein of the kit (reaction liquid volume: 100 μl; 16 to 20 hours). After the double-stranded RNA mixture of short strands (d-siRNA) cleaved by the dicer was confirmed in 3% agarose gel electrophoresis, desalination and removing of un-cleaved RNA were performed with the column attached to the kit, and finally d-siRNA of 22 bp was confirmed by agarose gel. Concentration was measured by absorption and it was adjusted to 5 μM with sterilized water and stored at −80° C. until it was used.

### EXAMPLE 7

### Transfection of siRNA (1)

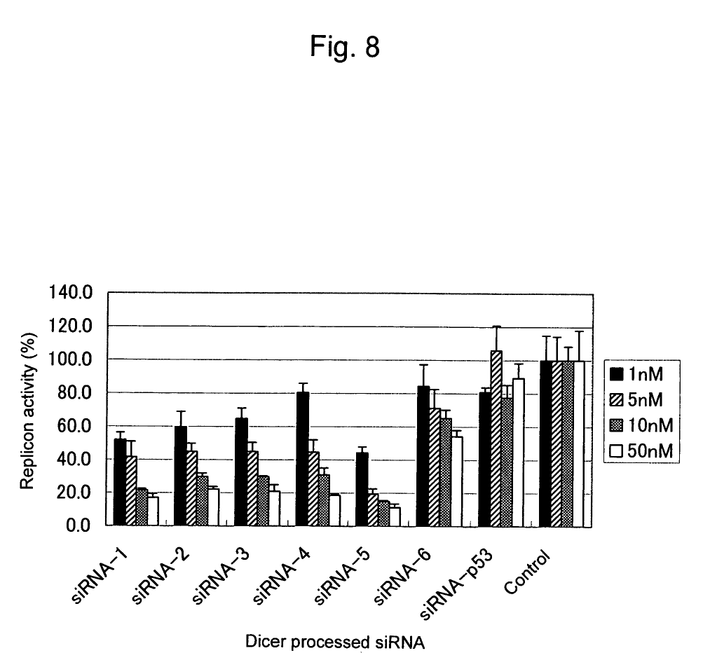

siRNAs prepared from double stranded precursor siRNA-1 to precursor siRNA-6 in Example 6 were introduced into the firefly luciferase HCV replicon cell (Huh-3-1) using the TransIT-TKO transfection reagent (Mirus Corporation cat. No. MIR2150) described in Example 4 in a concentration of 1 to 50 nM and luciferase activity was measured after 24 hours to determine the antivirotic activity. The results are shown in FIG. 8. In the drawing, siRNA-p53 shows the result of the case in which siRNA by dicer processing of the oncogene p53 was added, and the control shows the result in which sterilized water was added.

Consequently, it has been demonstrated that siRNA prepared by dicer inhibited the replicative activity of HCV replicon in concentration dependence and has an antivirotic activity.

### EXAMPLE 8

### Transfection of siRNA (2)

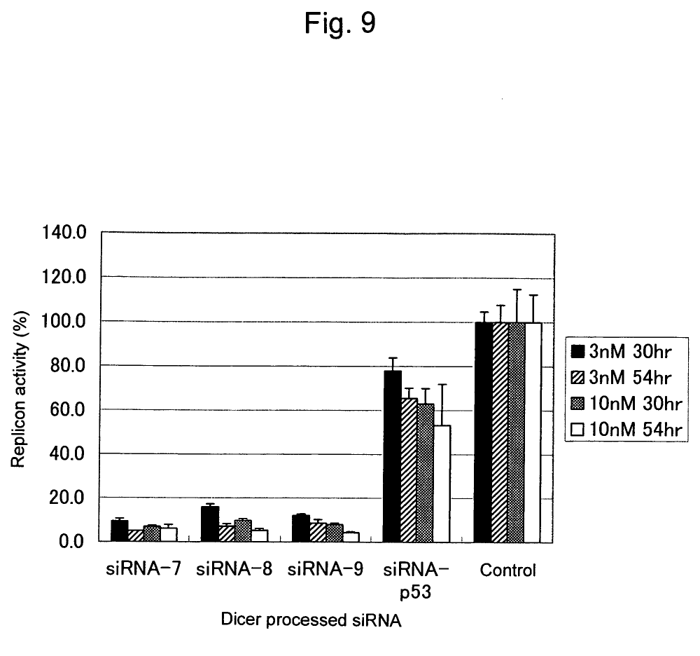

siRNAs prepared from double stranded precursor siRNA-7 to precursor siRNA-9 in Example 6 were introduced into the firefly luciferase HCV replicon cell (Huh-3-1) using the TransIT-TKO transfection reagent (Mirus Corporation cat. No. MIR2150) described in Example 4 in a concentration of 3 nM or 10 nM and luciferase activity was measured after 30 hours or 54 hours to determine the antivirotic activity. The results are shown in FIG. 9.

Consequently, it has been demonstrated that siRNA prepared by dicer inhibited the replicative activity of HCV replicon in concentration dependence and has an antivirotic activity as in Example 7.

### INDUSTRIAL APPLICABILITY

As described in full detail above, the present invention has provided an oligoribonucleotide or a peptide nucleic acid which sequence-specifically binds to the HCV-RNA and inhibits activities of HCV, and a therapeutic agent for hepatitis C which contains these components as an active ingredient and enabled to provide a new and definite therapeutic method of HCV.

It should be noted that all the publications, patents and patent applications cited in this specification are entirely incorporated in this specifications as reference.

1. An oligoribonucleotide or peptide nucleic acid which sequence-specifically binds to the RNA of a hepatitis C virus (HCV).

2. The oligoribonucleotide or peptide nucleic acid according to claim 1 which hybridizes with the RNA of HCV under stringent conditions.

3. The oligoribonucleotide or peptide nucleic acid according to claim 1 characterized in that the oligoribonucleotide or peptide nucleic acid hybridizes with the sequence of a 5′ non-coding region of the RNA of HCV.

4. The oligoribonucleotide or peptide nucleic acid according to claim 1 characterized in that the oligoribonucleotide or peptide nucleic acid hybridizes with the sequence of a highly identical region of the genetic sequences of a plurality of types of HCV different in genotype.

5. The oligoribonucleotide or peptide nucleic acid according to claim 1 which is a double-stranded RNA.

6. The oligoribonucleotide or peptide nucleic acid according to claim 1 which has a chain length of 19 to 23 bp.

7. An oligoribonucleotide having a nucleotide sequence shown in any one of SEQ ID Nos. 20 to 34.

8. An oligoribonucleotide which hybridizes under stringent conditions either with an RNA region of HCV having a sequence complementary to the oligoribonucleotide according to claim 7 or an RNA region of HCV hybridizing under stringent conditions with said oligoribonucleotide.

9. An oligoribonucleotide represented by a nucleotide sequence consisting of 19 to 23 contiguous bases in any one of the nucleotide sequences shown in SEQ ID Nos. 47 to 55.

10. An oligoribonucleotide which hybridizes under stringent conditions either with an RNA region of HCV having a sequence complementary to the oligoribonucleotide according to claim 9 or an RNA region of HCV hybridizing under stringent conditions with said oligoribonucleotide.

11. A vector which expresses the oligoribonucleotide according to claim 1.

12. A therapeutic agent for hepatitis C containing as an active ingredient the oligoribonucleotide or peptide nucleic acid according to claim 1.

13. A method of inhibiting replication ability of HCV by allowing the oligoribonucleotide or peptide nucleic acid according to claim 1 to bind to the HCV-RNA.

14. A vector which expresses the oligoribonucleotide according to claim 9.

15. A therapeutic agent for hepatitis C containing as an active ingredient the oligoribonucleotide or peptide nucleic acid according to claim 9.

16. A therapeutic agent for hepatitis C containing as an active ingredient the vector according to claim 11.

17. A method of inhibiting replication ability of HCV by allowing the oligoribonucleotide or peptide nucleic acid according to claim 9 to bind to the HCV-RNA.


SEQUENCE LISTING (56 sequences)

SEQ ID NO: 1 - DNA, 500 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaaaaaaaaacaaacgtaacaccaaccgtcgcccacaggacgtcaagttcccgggtggcggtcagatcgttggtggagtttacttgttgccgcgcaggggccctagattgggtgtgcgcgcgacgagaaagacttcc

SEQ ID NO: 2 - DNA, 500 bp, Hepatitis C virus
cgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgcccaggaagacttccgagcggtcg

SEQ ID NO: 3 - DNA, 500 bp, Hepatitis C virus
ttgggggcgacactccaccatagatcactcccctgtgaggaactactgtcttcacgcagaaagcgtctagccatggcgttagtatgagtgttgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacctcaaagaaaaaccaaacgtaacaccaaccgccgcccacaggacgttaagttcccgggcggtggtcagatcgttggtggagtttacctgttgccgcgcaggggccccaggttgggtgtgcgcgcgactaggaagacttccgagcggtcgcaa

SEQ ID NO: 4 - DNA, 500 bp, Hepatitis C virus
gggccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcccaaaccccaaagaaaaaccaaacgtaacaccaaccgtcgcccacaggacgtcaagttcccgggtggtggtcagatcgttggtggagtttacctgttgccgcgcaggggccccaggttgggtgtgcgcgcgactaggaagactt

SEQ ID NO: 5 - DNA, 500 bp, Hepatitis C virus
acccgccccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagaccgggtcctttcttggataaacccgctctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaacccaaagaaacactaaccgtcgcccacaagacgttaagtttccgggcggcggccagatcgttggcggagtatacttgttgccgcgtaggggccccagattgggtgtgcgcacagcaaggaagacttcg

SEQ ID NO: 6 - DNA, 500 bp, Hepatitis C virus
acccgccccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaacccacagaaacactaaccgtcgcccacaagacgttaagtttccgggcggcggccagatcgttggcggagtatacttgttgccgcgcaggggccctagattgggtgtgcgcacgacaaggaagacttcg

SEQ ID NO: 7 - DNA, 500 bp, Hepatitis C virus
acccgcccctaataggggcgacactccgccatgaaccactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggtcatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcgg

SEQ ID NO: 8 - DNA, 500 bp, Hepatitis C virus
acctgcccctaataggggcgacactccgccatgaatcactcccctgtgaggaactactgtcttcacgcagaaagcgcctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggcgcttgcgagtgccccgggaggtctcgtagaccgtgcaccatgagcacaaatcctaaacctcaaagaaaaaccaaaagaaacaccaaccgtcgcccagaagacgttaagttcccgggcggcggccagatcgttggcggagtatacttgttgccgcgcaggggccccaggttgggtgtgcgcacgacaaggaaaacttcgg

SEQ ID NO: 9 - DNA, 500 bp, Hepatitis C virus; identical to SEQ ID NO: 6

SEQ ID NO: 10 - DNA, 500 bp, Hepatitis C virus
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagaccgctagccgagtagcgttgggttgcgaaaggccttgtggtactgcctgatagggtgcttgcgagtgccccgggaggtctcgtagaccgtgcaccatgagcacaaatcctaaacctcaaagacaaaccaaaagaaacaccagccgtcgcccacaagacgttaggtttccgggcggcggccagatcgttggcggagtatacttgttgccgcgcaggggccccaggttgggtgtgcgcgcgacaaggaagacttcgg

SEQ ID NO: 11 - DNA, 500 bp, Hepatitis C virus
gcccgccccctgatgggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattaccggaaagactgggtcctttcttggataaacccactctatgtccggtcatttgggcacgcccccgcaagactgctagccgagtagcgttgggttgcgaaaggccttgtggtactgcctgatagggtgcttgcgagtgccccgggaggtctcgtagaccgtgcatcatgagcacaaatcctaaacctcaaagaaaaaccaaaagaaacacaaaccgccgcccacaggacgttaagttcccgggtggcggtcagatcgttggcggagtttacttgctgccgcgcaggggccccaggttgggtgtgcgcgcgacaaggaagacttct

SEQ ID NO: 12 - DNA, 311 bp, Hepatitis C virus
gcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgt

SEQ ID NO: 13 - DNA, 371 bp, Hepatitis C virus
gtccagctggttcgtggctggttacagcgggggagacatatatcacagcctgtctcgtgcccgaccccgctggttcatgttgtgcctactcctactttcagtaggggtaggcatctacctgctccccaaccgataaacggggagctaaacactccaggccaataggccatttctttttttttttttttttttttttctttttttttttttttttttttttttttttttttttttttttttctttcttttgttttttttttttttcttctttttggtggctccatcttagccctagtcacggctagctgtgaaaggtccgtgagccgcatgactgcagagagtgctgatactggcctctctgcagatcatgt

SEQ ID NO: 14 - DNA, 439 bp, Hepatitis C virus
tgggcggtgaagaccaagctcaaactcactccattgccggaagcgcgcctcctggatttatccagctggttcactgtcggcgccggcgggggcgacatttatcacagcgtgccgcgtgcccgaccccgcttattactccttggcctactcctactttttgtaggggtaggccttttcctactccccgctcggtagagcggcacacattagctacactccatagctaactgtccctttttttttgttttttttttttttttttttttttttttttcttttttttttttttttttgtttcttttccttctcatttccttcttatcttaattacttcctttcctggtggctccatcttagccctagtcacggctagctgtgaaaggtccgtgagccgcatgactgcagagattgccgtaactggtatctctgcagatcatgt

SEQ ID NO: 15 - DNA, 347 bp, Hepatitis C virus
cctggatttatccagctggttcactgtcggcgccggcgggggcgacatttatcacagcgtgccgcgtgcccgaccccgcttattactccttggcctactcctactttttgtaggggtaggccttttcctactccccgctcggtagagcggcacacattagctacactccatagctaactgtccctttttttttttttttttgtttcttttccttctcatttccttcttatcttaattactttctttcctggtggctccatcttagccctagtcacggctagctgtgaaaggtccgtgagccgcatgactgcagagattgccgtaactggtatctctgcagatcatgt

SEQ ID NO: 16 - DNA, 360 bp, Hepatitis C virus
tttatccagttggtttaccgtcggcgccggcgggggcgacatttatcacagcgtgtcgcgtgcccgaccccgcttattactccttagcctactcctacttttcgtaggggtaggcctctttttactccccgctcggtagagcggcacacattagctacactccatagctaactgttccttttttttttttttttttttttttttttttttttttttttttttttttttttttttttttttttttttttctttccttcctttctcaccttcttttacttctttcctggtggctccatcttagccctagtcacggctagctgtgaaaggtccgtgagccgcatgactgcagagagtgccgta

SEQ ID NO: 17 - DNA, 378 bp, Hepatitis C virus
ggacttatccagttggttcaccgtcggcgccggcgggggcgacatttttcacagcgtgtcgcgcgcccgaccccgctcattactcttcggcctactcctacttttcgtaggggtaggcctcttcctactccccgctcggtagagcggcacacactaggtacactccatagctaactgttcctttttttttttttttttttttttttttttttttttttttttttcttttttttttttttccctctttcttcccttctcatcttattctactttctttcttggtggctccatcttagccctagtcacggctagctgtgaaaggtccgtgagccgcatgactgcagagagtgccgtaactggtctctctgcagatcatgt

SEQ ID NO: 18 - DNA, 374 bp, Hepatitis C virus
ggatttgtccagttggtttaccgtcggcgccggcgggggcgacatttatcacagcgtgtcgcgtgcccgaccccgcctattactccttagcctactcctactttctgtaggggtaggcctcttcctactccccgctcgatagagcggcacacattagctacactccatagctaactgttccttttttttttttttttttttttttttttttttttttttctttttttttttttttccctctttcttcccttctcatcttattctactttctttcttggtggctccatcttagccctggtcacggctagctgtgaaaggtccgtgagccgcatgactgcagagagtgccgtaactggtctctctgcagatcatgt

SEQ ID NO: 19 - DNA, 354 bp, Hepatitis C virus
tagatttatccgggtggttcaccgtgggcgccggcgggggcgacatctttcacagcgtgtcgcatgcccgaccccgcctattactcctttgcctactcctacttagcgtaggagtaggcatctttttactccccgctcggtagagcggcaaaccctagctacactccatagctagttttcttttttttttttttttttttttttgttttttttttttttcctctttttccgtattttttttttttcctcttttcttggtggctccatcttagccctagtcacggctagctgtgaaaggtccgtgagccgcatgactgcagagagtgccgtaactggtctctctgcagatcatgt

SEQ ID NO: 20 - RNA, 21 nt, Artificial Sequence
  Description of Artificial Sequence 5′-UTR target siRNA
ggaacuacugucuucacgcag

SEQ ID NO: 21 - RNA, 21 nt, Artificial Sequence
  Description of Artificial Sequence 5′-UTR target siRNA
gccauaguggucugcggaacc

SEQ ID NO: 22 - RNA, 22 nt, Artificial Sequence
  Description of Artificial Sequence 5′-UTR target siRNA
aggccuugugguacugccugau

SEQ ID NO: 23 - RNA, 20 nt, Artificial Sequence
  Description of Artificial Sequence 5′-UTR target siRNA
gucucguagaccgugcauca

SEQ ID NO: 24 - DNA, 21 bp, Artificial Sequence
  Description of Artificial Sequence 5′-UTR target siRNA
gcgaaaggccttgtggtactg

SEQ ID NO: 25 - DNA, 20 bp, Artificial Sequence
  Description of Artificial Sequence 5′-UTR target siRNA
gtctcgtagaccgtgcacca

SEQ ID NO: 26 - DNA, 21 bp, Artificial Sequence
  Description of Artificial Sequence 5′-UTR target siRNA
gucucguagaccgugcaucat

SEQ ID NO: 27 - RNA, 21 nt, Artificial Sequence
  Description of Artificial Sequence 5′-UTR target siRNA
ggaacuacugucuucacgcag

SEQ ID NO: 28 - RNA, 21 nt, Artificial Sequence
  Description of Artificial Sequence 5′-UTR target siRNA
gccauaguggucugcggaacc

SEQ ID NO: 29 - RNA, 22 nt, Artificial Sequence
  Description of Artificial Sequence 5′-UTR target siRNA
aggccuugugguacugccugau

SEQ ID NO: 30 - RNA, 20 nt, Artificial Sequence
  Description of Artificial Sequence 5′-UTR target siRNA
gucucguagaccgugcauca

SEQ ID NO: 31 - DNA, 21 bp, Artificial Sequence
  Description of Artificial Sequence 5′-UTR target siRNA
gcgaaaggccttgtggtactg

SEQ ID NO: 32 - DNA, 20 bp, Artificial Sequence
  Description of Artificial Sequence 5′-UTR target siRNA
gtctcgtagaccgtgcacca

SEQ ID NO: 33 - DNA, 21 bp, Artificial Sequence
  Description of Artificial Sequence 3′-UTR target siRNA
ggctccatcttagccctagtc

SEQ ID NO: 34 - DNA, 21 bp, Artificial Sequence
  Description of Artificial Sequence 3′-UTR target siRNA
ggctagctgtgaaaggtccgt

SEQ ID NO: 35 - DNA, 25 bp, Artificial Sequence
  Description of Artificial Sequenceprimer Ds5-41-S25
actcccctgtgaggaactactgtct

SEQ ID NO: 36 - DNA, 25 bp, Artificial Sequence
  Description of Artificial Sequenceprimer Ds3-8864-S25
aggatgattctgatgacccatttct

SEQ ID NO: 37 - DNA, 23 bp, Artificial Sequence
  Description of Artificial Sequenceprimer Ds3-9267-S23
gcgggggagacatatatcacagc

SEQ ID NO: 38 - DNA, 25 bp, Artificial Sequence
  Description of Artificial Sequenceprimer Ds5-201-S25
tggatcaacccgctcaatgcctgga

SEQ ID NO: 39 - DNA, 25 bp, Artificial Sequence
  Description of Artificial Sequenceprimer Ds5-261-S25
tagtgttgggtcgcgaaaggccttg

SEQ ID NO: 40 - DNA, 25 bp, Artificial Sequence
  Description of Artificial Sequenceprimer Ds5-311-S25
gagtgccccgggaggtctcgtagac

SEQ ID NO: 41 - DNA, 23 bp, Artificial Sequence
  Description of Artificial Sequenceprimer Ds5-612-R23
ccctcgttgccatagaggggcca

SEQ ID NO: 42 - DNA, 25 bp, Artificial Sequence
  Description of Artificial Sequenceprimer Ds5-857-R25
aaccgggcaaattccctgttgcata

SEQ ID NO: 43 - DNA, 25 bp, Artificial Sequence
  Description of Artificial Sequenceprimer Ds3-9537-R25
gactagggctaagatggagccacca

SEQ ID NO: 44 - DNA, 23 bp, Artificial Sequence
  Description of Artificial Sequenceprimer Ds3-9611-R23
acatgatctgcagagaggccagt

SEQ ID NO: 45 - DNA, 23 bp, Artificial Sequence
  Description of Artificial Sequenceprimer Ds5-397-R23
gcggcggttggtgttacgtttgg

SEQ ID NO: 46 - DNA, 25 bp, Artificial Sequence
  Description of Artificial Sequenceprimer Ds5-360-R25
ttaggatttgtgctcatgatgcacg

SEQ ID NO: 47 - DNA, 572 bp, Artificial Sequence
  Description of Artificial SequencePCR product siRNA-1
actcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaaccccaaagaaaaaccaaacgtaacaccaaccgccgcccacaggacgtcaagttcccgggtggtggtcagatcgttggtggagtttacctgttgccgcgcaggggccccaggttgggtgtgcgcgcgactaggaagacttccgagcggtcacaacctcgtggaaggcgacaacctatccccaaggctcgccagcccgagggcagggcctgggctcagcccgggtacccttggcccctctatggcaacgaggg

SEQ ID NO: 48 - DNA, 817 bp, Artificial Sequence
  Description of Artificial SequencePCR product siRNA-2
actcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaaccccaaagaaaaaccaaacgtaacaccaaccgccgcccacaggacgtcaagttcccgggtggtggtcagatcgttggtggagtttacctgttgccgcgcaggggccccaggttgggtgtgcgcgcgactaggaagacttccgagcggtcacaacctcgtggaaggcgacaacctatccccaaggctcgccagcccgagggcagggcctgggctcagcccgggtacccttggcccctctatggcaacgagggcatggggtgggcaggatggctcctgtcaccccgcggctcccggcctagttggggccccacggacccccggcgtaggtcgcgtaatttgggtaaggtcatcgataccctcacatgcggcttcgccgacctcatggggtacattccgctcgtcggcgcccccctagggggcgttgccagggccctggcacatggtgtccgggttgtggaggacggcgtgaactatgcaacagggaatttgcccggtt

SEQ ID NO: 49 - DNA, 674 bp, Artificial Sequence
  Description of Artificial SequencePCR product siRNA-3
aggatgattctgatgacccatttcttctccatccttctagcccaggagcaacttgaaaaagccctggattgccagatctacggggcctgttactccattgagccacttgacctacctcagatcattgaacgactccatggtcttagcgcattttcactccatagttactctccaggtgagatcaatagggtggcttcatgcctcaggaaacttggggtaccacccttgcgagtctggagacatcgggccagaagtgtccgcgctaagctgctgtcccagggggggagggctgccacttgtggtaagtacctcttcaactgggcagtaaggaccaagctcaaactcactccaatcccggcagcgtcccagttggacttgtccagctggttcgtggctggttacagcgggggagacatatatcacagcctgtctcgtgcccgaccccgctggttcatgttgtgcctactcctactttcagtaggggtaggcatctacctgctccccaaccgataaacggggagctaaacactccaggccaataggccatttctttttttttttttttttttttttctttttttttttttttttttttttttttttttttttttttttttctttcttttgttttttttttttttcttctttttggtggctccatcttagccctagtc

SEQ ID NO: 50 - DNA, 748 bp, Artificial Sequence
  Description of Artificial SequencePCR product siRNA-4
aggatgattctgatgacccatttcttctccatccttctagcccaggagcaacttgaaaaagccctggattgccagatctacggggcctgttactccattgagccacttgacctacctcagatcattgaacgactccatggtcttagcgcattttcactccatagttactctccaggtgagatcaatagggtggcttcatgcctcaggaaacttggggtaccacccttgcgagtctggagacatcgggccagaagtgtccgcgctaagctgctgtcccagggggggagggctgccacttgtggtaagtacctcttcaactgggcagtaaggaccaagctcaaactcactccaatcccggcagcgtcccagttggacttgtccagctggttcgtggctggttacagcgggggagacatatatcacagcctgtctcgtgcccgaccccgctggttcatgttgtgcctactcctactttcagtaggggtaggcatctacctgctccccaaccgataaacggggagctaaacactccaggccaataggccatttctttttttttttttttttttttttctttttttttttttttttttttttttttttttttttttttttttctttcttttgttttttttttttttcttctttttggtggctccatcttagccctagtcacggctagctgtgaaaggtccgtgagccgcatgactgcagagagtgctgatactggcctctctgcagatcatgt

SEQ ID NO: 51 - DNA, 357 bp, Artificial Sequence
  Description of Artificial SequencePCR product siRNA-5
actcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaaccccaaagaaaaaccaaacgtaacaccaaccgccgc

SEQ ID NO: 52 - DNA, 345 bp, Artificial Sequence
  Description of Artificial SequencePCR product siRNA-6
gcgggggagacatatatcacagcctgtctcgtgcccgaccccgctggttcatgttgtgcctactcctactttcagtaggggtaggcatctacctgctccccaaccgataaacggggagctaaacactccaggccaataggccatttctttttttttttttttttttttttctttttttttttttttttttttttttttttttttttttttttttctttcttttgttttttttttttttcttctttttggtggctccatcttagccctagtcacggctagctgtgaaaggtccgtgagccgcatgactgcagagagtgctgatactggcctctctgcagatcatgt

SEQ ID NO: 53 - DNA, 197 bp, Artificial Sequence
  Description of Artificial SequencePCR product siRNA-7
tggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaaccccaaagaaaaaccaaacgtaacaccaaccgccgc

SEQ ID NO: 54 - DNA, 100 bp, Artificial Sequence
  Description of Artificial SequencePCR product siRNA-8
tagtgttgggtcgcgaaaggccttgtggtactgcctgatagggtgcttgcgagtgccccgggaggtctcgtagaccgtgcatcatgagcacaaatcctaa

SEQ ID NO: 55 - DNA, 50 bp, Artificial Sequence
  Description of Artificial SequencePCR product siRNA-9
gagtgccccgggaggtctcgtagaccgtgcatcatgagcacaaatcctaa

SEQ ID NO: 56 - DNA, 9611 bp, Hepatitis C virus
gggccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaaccccaaagaaaaaccaaacgtaacaccaaccgccgcccacaggacgtcaagttcccgggtggtggtcagatcgttggtggagtttacctgttgccgcgcaggggccccaggttgggtgtgcgcgcgactaggaagacttccgagcggtcacaacctcgtggaaggcgacaacctatccccaaggctcgccagcccgagggcagggcctgggctcagcccgggtacccttggcccctctatggcaacgagggcatggggtgggcaggatggctcctgtcaccccgcggctcccggcctagttggggccccacggacccccggcgtaggtcgcgtaatttgggtaaggtcatcgataccctcacatgcggcttcgccgacctcatggggtacattccgctcgtcggcgcccccctagggggcgttgccagggccctggcacatggtgtccgggttgtggaggacggcgtgaactatgcaacagggaatttgcccggttgctctttctctatcttcctcttggctctgctgtcctgtttgaccatcccagcttccgcttatgaggtgcgcaacgtatccgggatataccatgtcacgaacgactgctccaactcaagtattgtgtatgaggcagcggacatgatcatgcatacccccgggtgcgtgccctgcgttcgggagggcaactcctcccgttgctgggtggcacttactcccacgctagcggccaggaatgccagcgtccccactacggcaatacgacgccatgtcgatttgctcgttggggcggctgctttctgctccgctatgtatgtgggagatctctgcggatctgttttccttgtctcccagctgttcaccttctcgccccgccggcatgagacaatacaggactgcaattgctcaatctatcccggccacgtgtcaggtcaccgcatggcttgggacatgatgatgaactggtcgcctacaacggccctggtggtgtcgcagttactccggatcccacaagctatcgtggacatggtggcgggggctcactggggtgtcctagcgggccttgcctactattccatggtggggaactgggctaaggtattgattgtgatgctactttttgccggcgtcgacggggagacccgtgtgacaggggggcagatagccagaaatgcctactcgctcacgaccctcttttcatctgggtcggctcagaacatccagctcataaacaccaacggtagctggcacatcaacaggactgccctgaactgcaatgactccctcaacaccgggtttcttgccgcgctgttctacacgcacaagttcaacgcgtccggatgtccagagcgcttggccagctgccgccccattgacaagttcgatcaggggtggggtcccatcacttatgctgagcagggcggccaggaccagaggccttattgctggcactacgcacctaaaccatgtggtattgtatccgcgtcgaaggtgtgtggtccagtgtattgtttcaccccaagcccagttgtagtggggacgaccgatcggttcggtgtccctacgtatagctggggggagaatgagacagacgtgctgctccttaacaacacgcggccgccgcaaggcaactggttcggctgtacgtggatgaacggcactgggttcaccaagacatgcgggggccccccgtgtaacatcggggggggcggcaataacaccttgacctgccctacggactgtttccggaagcaccccgcggccacttacacaaaatgtggttcgggaccttggctgacacccaggtgcttggtagactacccatacaggctctggcactacccctgcactgccaactttaccatcttcaaggttaggatgtatgtagggggcgtggagcacaggctcgatgctgcatgcaattggacccgaggggaacgttgcaacttggaggatagggatagattggagctcagcccgctactgctgtctacaacagagtggcaggtgctgccctgttctttcaccaccctaccggctctgtccactggtttaattcatctccatcagaacatcgtggacgtgcaatacctgtacggtatagggtcggcagttgtttcctttgcaatcaaatgggactatatcgtgatacttttcctcctcctggcggacgcgcgcgtctgtgcctgcttgtggatgatgctgctgatagcccaggccgaggccgccttagaaaacctggtggtcctcaatgcggcgtccgtggccggagcgcatggcattctctccttccttgtgttcttctgtgccgcctggtacatcaagggcaagctggtccccggggcagcatatgctttctatggagtatggccgctgctcctgcttctgctggccttaccaccacgagcttacgctatggagcgggagatggctgcatcgtgcggaggcgcggtgtttgtaggtctggtactcttgactttgtcaccatactataaagagttcctcgccaggctcatatggtggttgcaatattttatcaccagagccgaggcgcacctgcaagtgtggatcccccccctcaacattcgggggggccgcgatgccatcatcctcctcgcgtgtgtagtccacccagagctaatctttgacatcaccaaactcctgctcgccatactcggtccgctcatggtgctccaggctagcataactcaagtgccgtacttcgtacgcgcccaagggctcattcgtgcatgcatgttggtgcggaaggtagccgggggccattatgtccaaatggcctttgtgaagctgaccgcactgacaggtacgtacgtttatgaccatctaactccactgcgggactgggcccacgcgggcctgcgagacctcgcggtggcagtagagcccgttgtcttctctgacatggagaccaaggtcatcacctggggggcagacaccgcagcgtgtggggacattatcttgggtctacctgtctccgcccgaaggggtagggagatacttctggggccggccgatagtcttgaagggcaggggtggcggctccttgctcccatcacggcctattcccaacagacgcggggcctacttggttgcatcatcactagcctcacaggccgggacaaaaaccaagtcgagggggaggttcaagtggtctccaccgcgacacaatccttcctggcgacctgcgtcaatggcgcgtgctggactgtcttccatggtgccggctcaaagaccttagctggcccaaaaggtccaatcacccagatgtacactaatgtagacctggacctcgtcggctggcaggcgccccccgggtcgcgttctctgacaccatgcacctgcggcagctcagacctctatttggtcacgagacatgctgatgtcattccggtgcgccggcggggcgacagtaggggaagcctactctctcccagacctgtctcctacttgaaaggctcctcgggtggtccgctgctctgcccttcgaggcacgctgtgggcatcttccgggctgctgtgtgcacccggggggttgcgaaggcggtggatttcatacccgttgaatcaatggaaactactatgcggtctccggtcttcacggataactcatcccccccggccgtaccgcagacattccaagtggcccatctacacgcccctactggcagcggcaagagcactaaggtgccggctgcatatgcagcccaagggtataaggtgctcgtcctgaacccgtccgttgccgctaccttgggttttggggcgtatatgtctaaggcacatggtatcgaccccaacatcagaactggggtaagggccatcaccacgggcgcccctattacatactccacctacggcaagttccttgccgacggcggttgttccgggggcgcctatgacatcataatatgtgatgagtgccactcaactgactcgactaccatcttgggcattggcacagtcctggaccaagcggagacggctggagcgcggctcgtcgtgctcgccaccgctacgcctccgggatcggtcaccgtgccacaccccaatattgaggaggtggccctgtccaacgctggagaaatccccttctacggcaaagccatccccattgaggtcatcaaggggggaagacatctcattttctgccattccaagaagaagtatgacgagctcgccgcaaagctatcagccctcggacttaatgctgtagcatattatcggggtcttgatgtgtccgtcataccgaccaacggagacgtcgttgtcgtggcaacagacgctctaatgacgggctttaccggcgactttgactcagtgatcgactgtaacacatgtgtcacccagacagtcgatttcagcctggatcccaccttcaccatcgagacgacgaccgtgccccaagacgcagtggcgcgatcacagcggcggggtaggactggtaggggcaggagaggcatctacaggtttgtgactccaggagaacggccctcgggcatgttcgattcctcggtcctgtgtgagtgctatgacgcgggctgtgcttggtacgagctcacgcctgctgagacctcggttaggttgcgggcttacctgaatacaccagggttgcccgtctgccaggaccatctggagttttgggagagcgtctccacaggcctcacccacatagatgcccattttctgtcccagactaaacaggcaggagacaacttcccctacctggtagcataccaagccacagtgtgcgccagagctcaagctccacctccatcatgggatcaaatgtggaagtgtctcatacggctcaaacccacgctgcacgggccaacacccctgctgtataggctaggagccgtccaaaatgagatcaccctcacacaccccatgaccaaattcatcatggcatgcatgtcggctgacctggaggtcgtcactagcacctgggtgctagtaggcggagtccttgcagctctggctgcatattgcttgacaacaggcagtgtggtcattgtgggtaggatcatcttgtccgggaggccggctgttattcccgacagggaagtcctctaccgggagttcgatgagatggaagagtgcgcctcacacctcccttacatcgaacagggaatgcagcttgccgagcaattcaagcagaaggcgctcggattgctgcaaacagccaccaagcaagcggaggctgctgctcccgtggtagaatccaagtggcgagcccttgagaccttctgggcgaagcacatgtggaatttcatcagcgggatacagtacctagcaggcttgtccactctgcctgggaaccccgcgatagcatcactgatggcattcacagcctctatcaccagcccgctctccacccagaataccctattatttaacatctgggggggatgggtggctgcccaactcgccccccccagtgctgcttcggctttcgtgggcgccggtatcgccggtgcggctgtcggcagcataggtcttgggaaggtgcttgtggacatcttggcgggatatggggcaggggtggctggcgcgctcgtagcttttaagatcatgagcggcgaggtgccctccaccgaggacctggttaacttactccctgccatcctctctcccggcgccctagtcgtcggggtcgtgtgcgcagcaatactgcgtcggcacgtgggcccgggagagggggctgtacagtggatgaaccggctgatagcgttcgcctcgcggggtaaccacgtttcccccgcgcactatgtgcctgagagcgacgctgcggcgcgtgttactcagatcctctccggccttaccatcactcagctgctgaagaggcttcaccactggatcaatgaggactgctccacgccatgctccggttcgtggctaagggatgtttgggactggatatgcacggtgttgactgacttcaagacctggctccagtccaagctcctgccgcggttaccgggggtccctttcttctcgtgtcaacgcgggtacaagggagtctggcggggggacggtatcatgcagaccacctgcccgtgtggagcacagatcaccggacatgtcaaaaacggttccatgaggatcgtcgggcctaaaacctgcagcagcacgtggcatggaacgttccccatcaacgcatacaccacaggcccatgcgcaccctccccggcgccaaactattccagggcgctatggcgggtggccgctgaggagtacgtggaggttacgcgggtgggggatttccactacgtgacgggcatgaccactgacaacgtaaagtgcccatgccaggttccggcccctgaattcttcactgaggtggatggagtgcggttgcacaggtacgctccggcgtgcaaacccctcctacgggaggaggtcacattccaggttgggctcaaccaatacctggttgggtcacagctcccatgcgagcccgaaccggatgtagcagtgctaacttccatgcttaccgacccctcccacatcacagcagagacggcaaagcgtaggctggctagggggtctcccccctccttggccagttcttcagctagccagttatctgcgccttccttgaaggcgacatgcactacccatcatgactccccggacgttgacctcatcgaggccaacctcctgtggcggcaggagatgggcgggaacatcacccgcgtggagtcagagaataaggtagtaattttggactctttcgatccgctccgagcggaggaggacgagagggaaccatccgttgcggcggagatcttgcggaaaaccaagaggttccccccggcgatgcccatatgggcacgcccggattacaaccctccgttgctagagtcctggaaagacccggactacgtccctccggtggtacacgggtgcccgctaccacctaccaaagctcctccgataccacccccacggagaaagaggacggtagtcctgacagagtccactgtgtcttctgccttggcggagcttgctactaagacctttggcagctccgggtcgtcggccgtcgacagcggcacggcaactgctcctcccgaccaggcttccgacgacggcgaccaaggatctgacgttgagtcgtattcctccatgccccctcttgagggagagccgggggaccccgatctcagcgacgggtcttggtctaccgtgagcgaggaggccggtgaggacgtcatctgctgctcaatgtcctacacatggacaggcgccttgatcacgccatgcgccgcggaggaaagcaagttgcccatcaacccgttgagcaactctttgttgcgtcaccacaacatggtctatgctacaacatcccgcagcgcaggcctacggcagaagaaggtcacctttgacagactgcaagtcctggacgaccactaccgggacgtgctcaaggagatgaaggcgaaggcgtccacagttaaggctaaactcctatccatagaagaagcctgtaagctgacgcccccacattcggccagatccaaatttggctatggggcaaaggacgtccggaacctatccagcaaggccgttaaccacatccgctccgtgtggaaggacttgctggaagacactgagacaccaattgacaccaccgtcatggcaaaaagtgaggttttctgcgtccaaccagagaaaggaggccgcaagccagctcgccttatcgtattcccagacttgggggttcgtgtatgcgagaagatggccctttatgacgtggtctccacccttcctcaggccgtgatgggctcctcatacggattccagtactcccctggacagcgggtcgagttcctggtgaatgcctggaaatcaaagaaatgccctatgggcttttcatatgacacccgctgttttgactcgacagtcactgagagtgacatccgtgttgaggagtcaatttaccaatgttgtgacttggcccccgaagccagacaggccataaagtcgctcacagagcggctttacattgggggtcccctgaccaattcaaaagggcagaactgtggctatcgccggtgccgcgcgagtggcgtgctgacgaccagctgcggtaatacccttacatgttacttgaaggcctctgcagcctgtcgagctgcaaagctccgggactgcacgatgctcgtgaacggagacgacctcgtcgtcatctgtgagagtgcgggaacccaagaggatgaggcgaacctacgagtcttcacggaggctatgactaggtattctgccccccccggggacccgccccgaccagaatacgacttggagctaataacatcatgttcctccaatgtgtcggtcgcgcacgatgcatctggcaaaagggtatactacctcacccgcgacccctccaccccccttgcacgggctgcgtgggagacagctagacacactccagttaattcctggctaggcaacatcattatgtatgcgcccaccttatgggcaaggatgattctgatgacccatttcttctccatccttctagcccaggagcaacttgaaaaagccctggattgccagatctacggggcctgttactccattgagccacttgacctacctcagatcattgaacgactccatggtcttagcgcattttcactccatagttactctccaggtgagatcaatagggtggcttcatgcctcaggaaacttggggtaccacccttgcgagtctggagacatcgggccagaagtgtccgcgctaagctgctgtcccagggggggagggctgccacttgtggtaagtacctcttcaactgggcagtaaggaccaagctcaaactcactccaatcccggcagcgtcccagttggacttgtccagctggttcgtggctggttacagcgggggagacatatatcacagcctgtctcgtgcccgaccccgctggttcatgttgtgcctactcctactttcagtaggggtaggcatctacctgctccccaaccgataaacggggagctaaacactccaggccaataggccatttctttttttttttttttttttttttctttttttttttttttttttttttttttttttttttttttttttctttcttttgttttttttttttttcttctttttggtggctccatcttagccctagtcacggctagctgtgaaaggtccgtgagccgcatgactgcagagagtgctgatactggcctctctgcagatcatgt